(19) United States
(12) Patent Application Publication
(10) Pub. No.: US 2020/0268015 A1
(43) Pub. Date: Aug. 27, 2020

(54) Method for hydrolysing protein materials

(71) Applicant: SLGK ZAKLAD BADAWCZO-WDROZENIOWY PROCESÓW CHEMICZNYCH SPÓLKA Z OGRANICZONA ODPOWEDZIALNOSCIA, BYDGOSZCZ (PL)

(72) Inventors: Henryk Lisaj, Ilawa (PL); Felix Samuel Sirovski, Canterbury (GB); Jerzy Stanislaw Grzesiak, Bydgoszcz (PL); Mieczyslaw Karwowski, Warsaw (PL)

(21) Appl. No.: 16/641,245

(22) PCT Filed: Sep. 8, 2018
(86) PCT No.: PCT/EP2018/074235

§ 371 Date: Feb. 23, 2020

(30) Foreign Application Priority Data
Sep. 10, 2017 (GB) 1714543.4

(51) Int. Cl. A23J 3/04; A23J 3/18; A23J 3/32; C07K 1/12

(52) U.S. Cl. CPC A23J 3/04; C07K 1/12; A23J 3/32; A23J 3/18

(57) ABSTRACT

A process for splitting protein materials of animal or plant origin to peptides with molecular weights from below 10 KDa to 260 KDa, decontamination of bone meal and destroying allergenic proteins. For these purposes the protein materials in an aqueous medium are subjected to the action of an alternate electric current at p H 7.5-9, temperature 30° C.-160° C. at a pressure of 1-6 bar and within a reaction time from 5 to 150 min. A protein splitting reactor comprising at least two electrodes contained within a cylindrical vessel having inlet and outlet ports at opposite ends; wherein one electrode is electrically and mechanically connected with the vessel wall and is a neutral point connected to earth; wherein the second electrode is electrically isolated from the first electrode and is connected to a single phase of an electric power supply.

### BACKGROUND

### a. Field of the Invention

Of many aspects of the present invention one relates to a process for the hydrolysis of protein materials of animal or plant origin.

Another aspect of the invention provides a process for destruction of such protein complexes as gluten that causes adverse reactions in humans for gluten containing foods.

Another aspect of the invention provides a method for manufacturing an animal feed from animal by-products by destruction of a prion protein that causes bovine spongiform encephalopathy (mad cow disease), scrapie in sheep, Creutzfeldt-Jacob Disease (CJD), Gerstman-Straussler Schemker (GSS) and Kuru in humans by decontamination of potentially prion containing material such as meat and bone meal and other animal by-products.

Yet another aspect of the present invention relates to a reactor for these processes.

### b. Related Art

Protein-containing plant and animal natural or waste products are known to be hydrolysed in a variety of ways splitting protein macromolecules both into amino acids and also peptides, comprising several amino acids.

This splitting can be carried out through the addition of acids or bases with the temperature effect.

The protein splitting can also be carried out enzymatically using proteases of different origin.

U.S. Pat. No. 9,034,402 describes a process for preparing a protein hydrolysate. The process comprises contacting a protein-containing material with endopeptidase that specifically cleaves peptide bonds of the protein material on the carboxyl terminal side of an arginine residue or a lysine residue to produce a protein hydrolysate composition. The obtained composition has a degree of hydrolysis of at least about 0.2% DH and a soluble solid index of at least about 80% at a pH greater than about 6.0.

U.S. Pat. No. 8,759,497 describes a process for preparing protein hydrolysates with defined molecular weight limits by splitting proteins in a temperature range from 140° C. to 250° C. at a pressure of 5 bar to 220 bar and reaction times up to 220 min. Molecules with different molecular weights in the magnitude of 10-50 KDa are obtained hereby as split products. Preferably splitting of protein materials is carried out in a temperature range between 180° C. and 220° C. and at a pressure between 50 and 75 bar and in reaction times from 25 to 40 min. The obtained protein hydrolysates have a molecular weight below 20 KDa.

U.S. Pat. No. 8,075,939 describes a method of manufacturing an animal feed by the following steps: (i) adding alkali to animal material to maintain pH of at least 8.5; (ii) heating the material at step (i) to a temperature in the range 55° C. to 99° C. and (iii) dehydrating the material produced at step (II). The method of invention is performed at about atmospheric pressure. The duration of steps (i) and (ii) is preferably 1-4 hours.

However, all the methods cited above have various disadvantages. Some of the methods require expensive enzymes and are not fully suitable for large scale production methods require quite drastic conditions and the use of corrosive reagents like hydrochloric or sulphuric acids and high pressures.

### SUMMARY OF THE INVENTION

Aspects of the invention are specified in the independent claims. Preferred features are specified in the dependent claims.

The object of the present invention is to provides a process by which it is possible to manufacture protein hydrolysates under mild conditions and without enzymatic steps.

Advantageously, the feedstock materials do not need to be dried as water is required for the process.

Among many applications, the process is suitable for converting animal and plant proteins into lower molecular weight peptides that could be used as animal feed. The process could be used also for complete destruction of harmful proteins like prions. Also the process could be used for the destruction of allergenic proteins like gluten, anti-nutrients present in soya meal, harmful plant glycosides like solanine present in plant meal, and genetically modified DNA in plant protein containing materials.

### BRIEF DESCRIPTION OF THE DRAWINGS

The present invention will now be further described, by way of example, with reference to the following figures, in which:

FIG. 1 is a flow chart showing the plant for manufacturing various protein hydrolysates of animal origin and prion destruction plant.

FIG. 2 is a flow chart showing the plant for manufacturing various protein hydrolysates of plant origin.

FIG. 3 shows the exemplary reactor schematics.

The drawings included in the present application are incorporated into, and form part of the specification. They illustrate embodiments of the present disclosure and, along with the description, serve to explain the principles of the disclosure. The drawings are only illustrative of certain embodiments and do not limit the disclosure.

### Definitions

As used herein, the term “animal”, as in “protein materials of animal origin” as described above, is intended to mean protein materials originated from mammals and non-mammals, including fish, molluscs, other cold-blooded animals, arthropods and prokaryotes.

As used herein, the term “plant” as in “protein materials of plant origin” as described above, is intended to mean protein materials originated from multi- or monocellular photosynthetic eukaryotes, other eukaryotes like yeasts, algi and fungi.

### DETAILED DESCRIPTION

The feedstock materials contain a large amount of protein material which can be converted into useful protein hydrolysates. The process of the present invention is concerned with a process of converting the feedstock into protein hydrolysates and decontaminated bone meal.

The most surprising and unexpected result was that passing alternate electric current through the slightly alkaline suspension of protein materials of animal or vegetable origin resulted in the quick formation of protein hydrolysates containing peptides with molecular weights depending on the amount of electrical energy applied to the material and individual amino acids. Depending on the residence time and amount of electrical energy applied the hydrolysates containing peptides with different molecular weights could be obtained. Elongated residence times result in the complete destruction of proteins and this method could be used for the manufacture of prion-free bone meal.

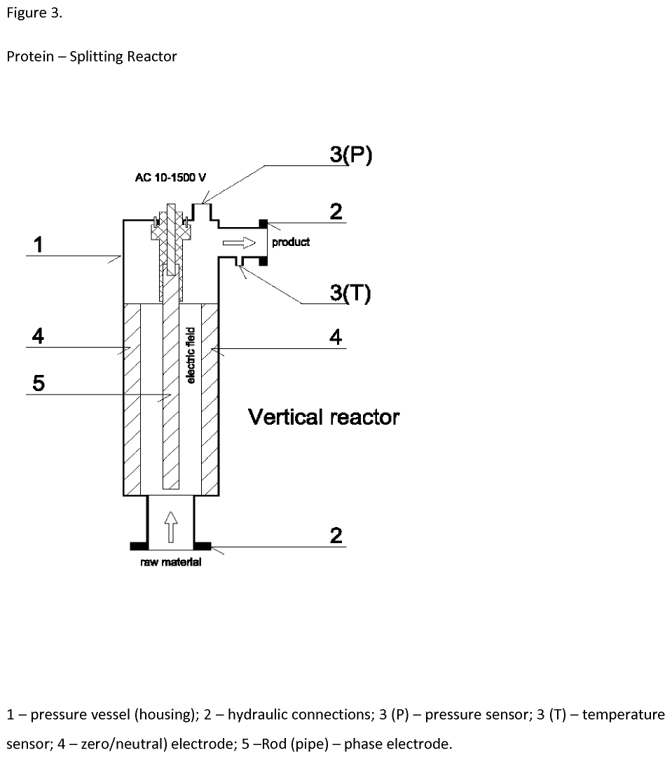

Ohmic heaters as described in the paper “A comprehensive review on applications of ohmic heating (OH)” by Mohamed Sakr, Shuli Liu published in Renewable and Sustainable Energy Reviews 39(2014) 262-269, DOI 10.1016/j.rser.2014.07.061 or elsewhere can be used as the protein-splitting reactor for passing electric current through the suspension of protein containing materials of animal or plant origin. However the preferable is the reactor shown in FIG. 3 and described further herein.

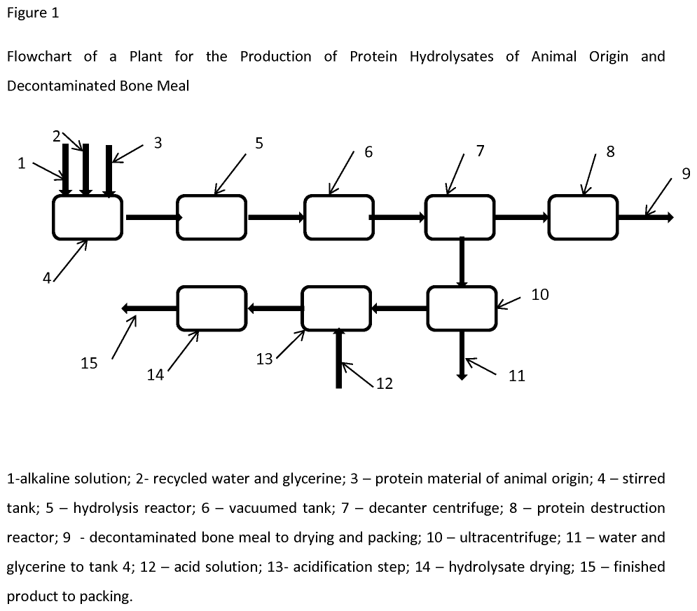

FIG. 1 shows the exemplary flow chart of a plant for the production of protein hydrolysates of animal origin and decontaminated bone meal.

Protein containing material of animal origin (meat and bone meal, feather meal, fish meal, etc.) is loaded into a stirred tank 4 where suspension of this material is prepared. The pH of obtained suspension is adjusted to pH 7.5-9.5, preferably 8-8.5 using any suitable inorganic water-soluble base, for example NaOH. Obtained alkaline suspension is pumped to a protein splitting reactor 5 where it is treated for 10-60 minutes at the temperature range of 35-150° C. and pressure up to 6 barg. From the reactor 5 the mixture is directed into a vacuum tank 6 where it is depressurised to the pressure of 0.1 bara. Depressurised reactor effluent is fed into a decanter centrifuge 7. Separated bone is fed into a second protein splitting reactor 8, where it is treated for 10-150 minutes at the temperature range of 120-150° C. and pressure up to 6 barg. After that reactor the decontaminated bone meal is directed to drying and packing.

Liquid stream from decanter centrifuge 7 is fed into ultracentrifuge 10 where protein hydrolysate is separated from water containing some glycerine. This water is returned to the front end to the stirred tank 4 where it used for the preparation of the suspension of protein containing material.

Obtained finished hydrolysate is neutralised with a suitable mineral acid in a stirred tank 14 and is directed 15 to drying (if necessary) and packing.

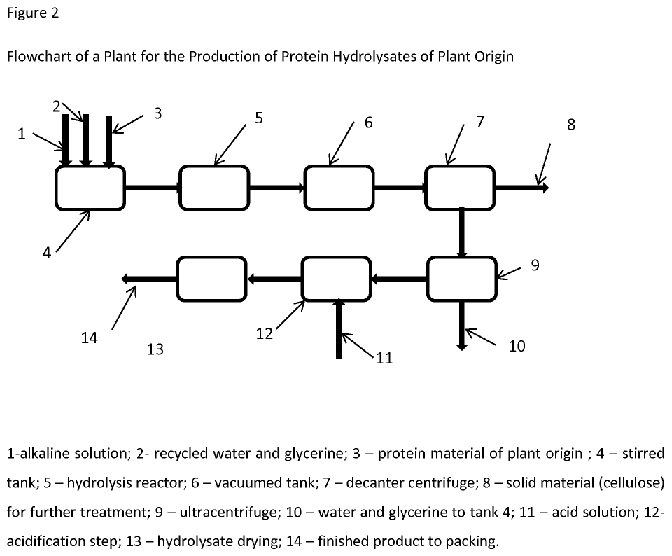

FIG. 2 shows the exemplary flow chart of a plant for the production of protein hydrolysates of plant origin.

Protein containing material of plant origin (lupine, soya, peas, wheat meal etc.) is loaded into a stirred tank 4 where suspension of this material is prepared. The pH of obtained suspension is adjusted to pH 7.5-9.5, preferably 8-8.5 using any suitable inorganic water-soluble base, for example NaOH. Obtained alkaline suspension is pumped to a protein-splitting reactor 5 where it is treated for 10-60 minutes at the temperature range of 35-150° C. and pressure up to 6 barg. From the reactor 5 the mixture is directed into a vacuum tank 6 where it is depressurised to the pressure of 0.1 bara. Depressurised reactor effluent is fed into a decanter centrifuge 7. Separated cellulose material is directed to further treatment.

Liquid stream from decanter centrifuge 7 is fed into ultracentrifuge 9 where protein hydrolysate is separated from water containing some glycerine. This water is returned to the front end to the stirred tank 4 where it used for the preparation of the suspension of protein containing material.

Obtained finished hydrolysate is neutralised with a suitable mineral acid in a stirred tank 12 and is directed to drying 13 (if necessary) and packing.

The process is preferably carried out in a continuous manner, for example, by pumping the feedstock reaction mixture through a sufficiently long ohmic heater or the reactor described below. Alternatively, it can be carried out as a batch process.

Instead of a conventional ohmic heater described elsewhere the preferred construction of the protein splitting reactor is described below.

The exemplary reactor chart is shown in FIG. 3.

The reactor consists of a pressure vessel 1 that could be manufactured from a suitable material required to withstand pressure, (for example stainless steel 316L) with hydraulic connections 2 (in case of a continuous process) and sensors for pressure and temperature, 3. Inside this casing is a tube 4 made of electrically conductive material (steel, graphite or other analogous suitable material) connected mechanically and electrically to the casing. The diameter and length of the tube is determined by the protein material conversion requirements. The tube 4 is a zero electrode. In the center of the reactor is placed the phase electrode 5 made of electrically conductive material (steel, graphite or other suitable material). This electrode could be a rod or a hollow pipe cooled by the converted material. This electrode is electrically isolated from the housing and internal graphite tube. For the reactor operations requiring power greater than 20 kW, a three-reactor system in parallel or in series configuration is used in order to ensure a uniform load on the network.

It will be appreciated that any suitable techniques may be employed in the process; for example instead of a decanter centrifuge a filter press, dewatering cyclone or a rotary drum vacuum filter can be used. Suitable device will be well known per se to those skilled in the art.

It should be understood, however, that the intention is not to limit the invention to the particular embodiments described. On the contrary, the intention is to cover all modifications, equivalents, and alternatives falling within the spirit and scope of the invention.

[symbol]

The invention is illustrated by the following examples. The examples are intended for illustration only and the present invention is not any way limited by the examples.

### Example 1

Lupine meal (3 kg) was loaded to the reactor that was described above. Then the reactor was sealed and the process started. The current frequency was 50 Hz.

The process parameters are listed in the table below.

| Time, hr: | Current, | Temperature | Pressure, | |
|---|---|---|---|---|
| min | A | [° C.] | barg | Notes |
| 00:07 | 30.0 | 81 | 0.00 | |
| 00:08 | 31.0 | 85 | 0.00 | |
| 00:09 | 34.0 | 94 | 0.00 | |
| 00:10 | 35.8 | 101 | 0.75 | |
| 00:11 | 39.4 | 108 | 1.30 | |
| 00:12 | 36.5 | 110 | 1.60 | |
| 00:13 | 38.2 | 112 | 1.75 | |
| 00:14 | 38.3 | 114 | 2.00 | |
| 00:15 | 38.4 | 116 | 2.30 | |
| 00:17 | 38.3 | 117 | 2.50 | |
| | | 117 | 2.50 | Stopped |
| 00:18 | 38.0 | 119 | 2.40 | |
| | | 119 | 2.40 | Stopped |
| 00:19 | 38.4 | 130 | 2.60 | |
| 00:20 | 0.0 | 133 | 2.60 | |
| | | 133 | 2.60 | First sample |
| | | | | taken |
| 00:25 | 33.0 | 134 | 2.50 | |
| 00:26 | 35.0 | 140 | 2.90 | |
| | | 140 | 2.90 | Stopped |
| | | 140 | 2.90 | Second sample |
| | | | | taken |
| 00:31 | 30.7 | 137 | 2.90 | |
| 00:32 | 30.7 | 137 | 2.90 | |
| 00:33 | 31.3 | 139 | 3.00 | |
| 00:34 | 32.7 | 140 | 3.30 | |
| 00:35 | 33.0 | 140 | 3.30 | Stopped |
| 00:35 | | 143 | 3.50 | Stopped |
| 00:37 | | 143 | 3.50 | Third sample |
| | | | | taken |
| 00:39 | 24.1 | 143 | 3.50 | |
| 00:40 | 24.9 | 134 | 2.50 | |
| 00:41 | 26.5 | 138 | 2.90 | |
| 00:42 | 26.9 | 141 | 3.30 | |
| 00:42 | | 143 | 3.60 | Stopped |
| 00:43 | 26.4 | 143 | 3.60 | |
| 00:44 | 27.3 | 144 | 3.50 | |
| 00:44 | | 147 | 4.00 | Stopped |
| 00:45 | | 147 | 4.00 | |
| | | 147 | 4.00 | Fourth sample |
| | | | | taken through |
| | | | | a bottom valve |

The collected hydrolysate was separated from the sediment by gravity separation. The analysis showed that hydrolysate contains peptides with molecular mass in the range of 60-240 KDa.

### Example 2

The hydrolysis was carried out as in the example 1 aside from current frequency that was 60 Hz. The hydrolysed material was 3 kg of a pea meal.

The process parameters are listed in the table below.

| Time, hr: | Current, | Temperature, | Pressure, |
|---|---|---|---|
| min | A | ° C. | barg |
| 00:00 | 35.0 | 22 | 0.00 |
| 00:02 | 34.3 | 24 | 0.00 |
| 00:03 | 27.9 | 29 | 0.00 |
| 00:05 | 28.0 | 34 | 0.00 |
| 00:06 | 27.8 | 38 | 0.00 |
| 00:07 | 34.7 | 41 | 0.00 |
| 00:08 | 28.1 | 45 | 0.00 |
| 00:10 | 28.0 | 49 | 0.00 |
| 00:11 | 22.2 | 52 | 0.00 |
| 00:13 | 22.0 | 54 | 0.00 |
| 00:15 | 21.9 | 56 | 0.00 |
| 00:16 | 21.9 | 57 | 0.00 |
| 00:20 | 29.1 | 59 | 0.00 |
| 00:21 | 28.1 | 61 | 0.00 |
| 00:22 | 28.1 | 62 | 0.00 |
| 00:25 | 28.2 | 67 | 0.00 |
| 00:36 | 28.3 | 73 | 0.00 |
| 00:38 | 28.3 | 75 | 0.25 |
| 00:39 | 28.3 | 77 | 0.00 |
| 01:04 | 21.8 | 92 | 0.00 |
| 01:09 | 28.6 | 97 | 0.50 |
| 01:30 | 32.9 | 96 | 0.00 |
| 01:31 | 33.1 | 97 | 0.00 |
| 01:32 | 33.1 | 98 | 0.00 |
| 01:33 | 33.1 | 100 | 0.10 |
| 01:40 | 33.1 | 104 | 0.50 |
| 01:41 | 33.2 | 105 | 0.55 |
| 01:43 | 33.1 | 106 | 0.60 |
| 01:44 | 33.2 | 107 | 0.65 |
| 01:45 | 32.8 | 108 | 0.70 |

The collected hydrolysate was separated from the sediment by gravity separation. The analysis showed that hydrolysate contains peptides with molecular mass in the range of 40-80 KDa.

### Example 3

The hydrolysis was carried out as in the example 1. The hydrolysed material was 3 kg of a soya meal.

The process parameters are listed in the table below.

| Time, hr: | Current, | Temperature, | Pressure, | |
|---|---|---|---|---|
| min | A | ° C. | barg | Notes |
| 00:00 | 35.2 | 23 | 0.00 | |
| 00:01 | 36.6 | 27 | 0.00 | |
| 00:02 | 36.8 | 31 | 0.00 | |
| 00:03 | 36.6 | 36 | 0.00 | |
| 00:04 | 36.8 | 41 | 0.00 | |
| 00:05 | 32.8 | 47 | 0.00 | |
| 00:06 | 14.2 | 51 | 0.00 | |
| 00:07 | 13.0 | 53 | 0.20 | |
| 00:08 | 9.8 | 55 | 0.50 | |
| 00:09 | 10.4 | 58 | 0.90 | |
| 00:10 | 7.7 | 59 | 0.80 | |
| 00:11 | 4.6 | 62 | 0.00 | Gas drained |
| 00:13 | 3.7 | 67 | 0.00 | |
| 00:14 | 3.3 | 68 | 0.00 | |
| 00:15 | 3.0 | 69 | 0.00 | |
| 00:16 | 3.6 | 70 | 0.00 | |
| 00:17 | 2.9 | 70 | 0.00 | |
| 00:18 | 2.7 | 70 | 0.00 | |
| 00:21 | 2.6 | 78 | 0.00 | |
| 00:23 | 6.7 | 97 | 1.40 | |
| 00:24 | 4.5 | 99 | 1.50 | |
| 00:25 | 3.5 | 101 | 1.40 | |
| 00:26 | 3.3 | 102 | 1.30 | |
| 00:27 | 2.8 | 102 | 1.20 | |
| 00:28 | 4.0 | 102 | 1.10 | |
| 00:29 | 5.9 | 104 | 1.20 | |
| 00:30 | 3.7 | 106 | 1.10 | |
| 00:32 | 10.0 | 106 | 1.00 | |
| 00:33 | 3.1 | 106 | 1.00 | |
| 00:35 | 3.2 | 107 | 1.00 | |
| 00:36 | 2.2 | 107 | 1.00 | |
| 00:38 | | | | First sample |
| | | | | taken |
| 00:44 | 2.5 | 125 | 2.50 | |
| 00:44 | 0.0 | 125 | 2.50 | Second sample |
| | | | | taken |
| 00:44 | 0.0 | 125 | 2.50 | Third sample |
| | | | | taken from |
| | | | | bottom valve |

The collected hydrolysate was separated from the sediment by gravity separation. The analysis showed that hydrolysate contains peptides with molecular mass in the range of 130-270 KDa.

### Example 4

The hydrolysis was carried out as in the example 1. The hydrolysed material was 3 kg of an animal by-product category 3 (meat and bone meal).

The process parameters are listed in the table below.

| Time, | Current, | Temperature, | Pressure, |
|---|---|---|---|
| hh:min | A | ° C. | barg |
| 00:00 | 35.0 | 22 | 0.00 |
| 00:02 | 34.3 | 24 | 0.00 |
| 00:03 | 27.9 | 29 | 0.00 |
| 00:05 | 28.0 | 34 | 0.00 |
| 00:06 | 27.8 | 38 | 0.00 |
| 00:07 | 34.7 | 41 | 0.00 |
| 00:08 | 28.1 | 45 | 0.00 |
| 00:10 | 28.0 | 49 | 0.00 |
| 00:11 | 22.2 | 52 | 0.00 |
| 00:13 | 22.0 | 54 | 0.00 |
| 00:15 | 21.9 | 56 | 0.00 |
| 00:16 | 21.9 | 57 | 0.00 |
| 00:20 | 29.1 | 59 | 0.00 |
| 00:21 | 28.1 | 61 | 0.00 |
| 00:22 | 28.1 | 62 | 0.00 |
| 00:25 | 28.2 | 67 | 0.00 |
| 00:36 | 28.3 | 73 | 0.00 |
| 00:38 | 28.3 | 75 | 0.25 |
| 00:39 | 28.3 | 77 | 0.00 |
| 01:04 | 21.8 | 92 | 0.00 |
| 01:09 | 28.6 | 97 | 0.50 |
| 01:30 | 32.9 | 96 | 0.00 |
| 01:31 | 33.1 | 97 | 0.00 |
| 01:32 | 33.1 | 98 | 0.00 |
| 01:33 | 33.1 | 100 | 0.10 |
| 01:40 | 33.1 | 104 | 0.50 |
| 01:41 | 33.2 | 105 | 0.55 |
| 01:43 | 33.1 | 106 | 0.60 |
| 01:44 | 33.2 | 107 | 0.65 |
| 01:45 | 32.8 | 108 | 0.70 |
| 01:47 | 32.7 | 109 | 0.75 |
| 01:48 | 32.8 | 110 | 0.80 |
| 01:49 | 32.7 | 112 | 0.80 |
| 01:50 | 32.9 | 113 | 0.90 |
| 01:53 | 32.9 | 114 | 1.00 |
| 01:54 | 33.0 | 115 | 1.10 |
| 01:55 | 33.0 | 116 | 1.10 |
| 01:57 | 33.0 | 117 | 1.15 |
| 02:01 | 26.0 | 119 | 1.20 |
| 02:07 | 32.9 | 120 | 1.30 |
| 02:09 | 32.9 | 121 | 1.40 |
| 02:12 | 32.6 | 122 | 1.40 |
| 02:13 | 39.2 | 123 | 1.50 |
| 02:15 | 33.0 | 124 | 1.50 |
| 02:20 | 36.2 | 125 | 1.50 |
| 02:23 | 35.2 | 126 | 1.60 |

The collected hydrolysate was separated from the sediment by gravity separation. The analysis showed that no peptides with molecular mass exceeding 10 KDa are present.

Hydrolysate also contained following amino acids determined by HPLC:

| Amino acid | Concentration, g/kg |
|---|---|
| Histidine | 2.44 ± 0.32 |
| Serine | 6.52 ± 0.85 |
| Arginine | 9.96 ± 1.29 |
| Glycine | 22.3 ± 1.8 |
| Asparagine | 10.7 ± 1.4 |
| Glutamine | 18.6 ± 2.4 |
| Threonine | 4.4 ± 0.57 |
| Alanine | 11.7 ± 1.6 |
| Proline | 14.2 ± 1.3 |
| Lysine | 6.22 ± 0.56 |
| Tyrosine | 2.74 ± 0.38 |
| Valine | 6.13 ± 0.67 |
| Isoleucine | 4.11 ± 0.49 |
| Leucine | 8.41 ± 0.93 |
| Phenylalanine | 4.82 ± 0.63 |
| Cysteine | 0.764 ± 0.099 |
| Methionine | 1.77 ± 0.25 |
| Tryptophan | 0.47 ± 0.06 |

### Example 5

The hydrolysis was carried out as in the example 1. The hydrolysed material was 3 kg of an animal by-product category 3 (poultry meal).

The process parameters are listed in the table below.

| Time. | Current. | Temperature. | Pressure. | |
|---|---|---|---|---|
| hr: min | A | ° C. | barg | Notes |
| 00:00 | 38.1 | | 0.00 | |
| 00:01 | 38.6 | 33 | 0.00 | |
| 00:02 | 39.1 | 36 | 0.00 | |
| 00:03 | 39.3 | 40 | 0.00 | |
| 00:04 | 39.0 | 42 | 0.00 | |
| 00:05 | 39.0 | 43 | 0.00 | |
| 00:06 | 39.4 | 48 | 0.00 | |
| 00:07 | 39.5 | 50 | 0.00 | |
| 00:08 | 39.5 | 52 | 0.00 | |
| 00:09 | 39.5 | 54 | 0.00 | |
| 00:10 | 39.8 | 56 | 0.00 | |
| 00:11 | 39.9 | 60 | 0.00 | |
| 00:13 | 40.0 | 65 | 0.00 | |
| 00:14 | 40.0 | 66 | 0.00 | |
| 00:15 | 40.0 | 67 | 0.00 | |
| 00:16 | 39.9 | 70 | 0.00 | |
| 00:17 | 40.0 | 73 | 0.00 | |
| 00:18 | 40.0 | 75 | 0.00 | |
| 00:19 | 39.7 | 76 | 0.00 | |
| 00:20 | 39.6 | 78 | 0.00 | |
| 00:21 | 39.5 | 80 | 0.00 | |
| 00:22 | 39.3 | 81 | 0.00 | |
| 00:23 | 39.1 | 81 | 0.00 | |
| 00:24 | 45.0 | 82 | 0.10 | |
| 00:25 | 45.3 | 83 | 0.25 | |
| 00:26 | 45.1 | 86 | 0.50 | |
| 00:27 | 45.6 | 89 | 0.60 | |
| 00:28 | 45.6 | 92 | 0.80 | |
| 00:29 | 45.6 | 94 | 1.00 | |
| 00:30 | 45.3 | 102 | 1.20 | |
| 00:31 | 45.6 | 106 | 1.30 | |
| 00:32 | 45.4 | 110 | 1.50 | |
| 00:33 | 45.0 | 113 | 1.60 | |
| 00:33 | | | | Sample |
| | | | | #1 |
| 00:34 | 45.1 | 123 | 1.70 | |
| 00:35 | 45.0 | 126 | 1.80 | |
| 00:35 | 0.0 | | | Stopped |
| 00:36 | 46.4 | 126 | 1.70 | |
| 00:37 | 45.7 | 127 | 1.90 | |
| 00:38 | 45.3 | 129 | 2.00 | |
| 00:39 | 45.0 | 130 | 2.00 | |
| 00:39 | | | | Sample |
| | | | | #2 |
| 00:40 | 46.2 | 130 | 2.00 | |
| 00:41 | 44.6 | 132 | 2.20 | |
| 00:42 | 44.7 | 136 | 2.60 | |
| 00:43 | 44.7 | 138 | 2.80 | |
| 00:44 | 44.8 | 140 | 3.00 | |
| 00:44 | 0.0 | | | Stopped |
| 00:46 | 0.0 | 140 | 2.60 | |
| 00:47 | 46.4 | 139 | 2.50 | |
| 00:48 | 45.6 | 140 | 2.90 | |
| 00:48 | 0.0 | | | Sample |
| | | | | #3 |
| 00:49 | 0.0 | | | Sample # |
| | | | | from |
| | | | | bottom |
| | | | | valve |

The collected hydrolysate was separated from the sediment by gravity separation. The analysis showed that hydrolysate contains peptides with molecular mass in the range of 110-230 KDa.

### Example 6

The hydrolysis was carried out as in the example 1. The hydrolysed material was 1.5 kg of a dried feather meal that was mixed with water in 1:1 ratio.

The process parameters are listed in the table below.

| Time, | | | | |
|---|---|---|---|---|
| hr: | Current, | Temperature, | Pressure, | |
| min | A | ° C. | barg | Notes |
| 00:00 | 29.0 | 30 | 0.00 | |
| 00:01 | 36.0 | 36 | 0.00 | |
| 00:02 | 37.7 | 40 | 0.00 | |
| 00:03 | 42.3 | 50 | 0.00 | |
| 00:04 | 42.0 | 58 | 0.00 | |
| 00:05 | 40.7 | 70 | 0.00 | |
| 00:06 | 40.2 | 79 | 0.30 | |
| 00:07 | 40.5 | 83 | 0.60 | |
| 00:08 | 40.5 | 90 | 1.00 | |
| 00:09 | 40.2 | 95 | 1.30 | |
| 00:10 | 39.7 | 108 | 1.60 | |
| 00:11 | 0.0 | | | Stopped |
| 00:12 | 0.0 | 116 | 1.50 | |
| 00:13 | 0.0 | 120 | 1.30 | |
| 00:14 | 0.0 | 121 | 1.20 | |
| 00:15 | 0.0 | | | |
| 00:16 | 0.0 | | | |
| 00:17 | | | | Started |
| 00:18 | 34.0 | 118 | 1.10 | |
| 00:19 | 33.3 | 120 | 1.30 | |
| 00:20 | 33.2 | 125 | 1.60 | |
| 00:21 | 33.3 | 128 | 1.90 | |
| 00:22 | 33.4 | 131 | 2.10 | |
| 00:23 | 33.8 | 134 | 2.50 | |
| 00:24 | 34.1 | 138 | 2.80 | |
| 00:25 | 34.1 | 140 | 3.00 | |
| 00:26 | 35.0 | 140 | 3.10 | Stopped |
| 00:27 | 35.0 | 141 | 3.20 | |
| 00:28 | 35.1 | 144 | 3.60 | |
| 00:29 | 35.5 | 146 | 3.80 | |
| 00:30 | 35.6 | 148 | 3.95 | |
| 00:31 | 37.2 | 149 | 4.00 | Stopped |
| 00:32 | 36.3 | 150 | 4.00 | Sample |
| | | | | taken |

The collected hydrolysate was separated from the sediment by gravity separation. The collected hydrolysate was separated from the sediment by gravity separation. The analysis showed that hydrolysate contains peptides with molecular mass in the range of 120-260 KDa

### Example 7

The hydrolysis was carried out as in the example 1. The hydrolysed material was 3 kg of a wet feather meal.

The process parameters are listed in the table below.

| Time, | Current, | Temperature, | Pressure, | |
|---|---|---|---|---|
| hr:min | A | ° C. | barg | Notes |
| 00:00 | 30.8 | | 0.00 | |
| 00:02 | 32.8 | 33 | 0.00 | |
| 00:03 | 35.0 | 42 | 0.00 | |
| 00:06 | 36.8 | 51 | 0.00 | |
| 00:12 | 38.1 | 76 | 1.00 | |
| 00:13 | 38.5 | 79 | 1.00 | |
| 00:14 | 38.4 | 81 | 1.10 | |
| 00:15 | 38.9 | 91 | 1.50 | |
| 00:17 | 39.1 | 97 | 1.70 | |
| 00:18 | 39.3 | 103 | 2.00 | |
| 00:19 | 39.4 | 105 | 2.00 | |
| 00:21 | 39.9 | 115 | 2.40 | |
| 00:25 | 40.0 | 131 | 3.00 | |
| 00:27 | 39.6 | 137 | 3.50 | |
| 00:28 | 40.0 | 140 | 3.70 | |
| 00:29 | 39.8 | 146 | 3.80 | |
| 00:29 | 0.0 | | | Stopped |
| 00:31 | 41.1 | 144 | 3.50 | |
| 00:32 | 40.4 | 145 | 3.90 | |
| 00:33 | 40.2 | 147 | 4.10 | |
| 00:34 | 40.5 | 150 | 4.10 | |
| 00:34 | 0.0 | | | Stopped |
| 00:39 | 40.9 | 140 | 3.10 | |
| 00:40 | 53.8 | 142 | 3.60 | Current |
| | | | | settings |
| | | | | changed |
| 00:41 | 53.6 | 145 | 3.80 | |
| 00:42 | 54.7 | 147 | 4.10 | |
| 00:42 | 54.5 | 150 | 4.40 | |
| 00:42 | 0.0 | | | Stopped |
| 00:42 | 55.1 | 151 | 4.00 | |
| 01:03 | 55.4 | 152 | 4.50 | |

The collected hydrolysate was separated from the sediment by gravity separation. The collected hydrolysate was separated from the sediment by gravity separation. The analysis showed that hydrolysate contains peptides with molecular mass in the range of 40-180 KDa.

1. A process for splitting protein materials of animal or plant origin, the process comprising:
mixing the protein material to a pourable suspension with water;
adjusting pH of the suspension above pH 7 and below pH 10 by adding alkaline material;
splitting of protein material in the suspension by passing alternate electrical current through the material, wherein the said step of splitting occurs in a protein splitting reactor;
separating the suspension with the split protein material into a sediment that contains the insoluble components of protein containing materials and an aqueous supernatant in which the products of split animal or plant proteins are dissolved;
isolating products of splitting animal or plant protein molecules so that the split protein molecules have defined molecular weights in the range of up to 260 KDa.

2. A process for splitting protein materials as specified in claim 1; wherein the pourable suspension has concentration of 5-50% mass of protein material.

3. A process for splitting protein materials as specified in claims 1-2; wherein the pourable suspension has pH in the range of 7.5-9, preferably 7.8-8.5 adjusted by adding alkaline material.

4. A process for splitting protein materials as specified in claims 1-3; wherein splitting occurs in a temperature range of 35° C. to 160° C. at a pressure of 1-6 bar;

5. A process for splitting protein materials as specified in claim 1-4, wherein within a reaction time from 5 to 150 min.

6. A process for splitting protein materials as specified in claim 1, wherein splitting is carried out in a continuous mode.

7. A process for splitting protein materials as specified in claim 1 or claim 6, wherein the protein splitting reactor is an ohmic heater.

8. A process for decontamination of bone meal from pathogenic protein agents by splitting protein materials as specified in claims 1-5, wherein in order to decontaminate bone meal the separated insoluble material is treated in the protein splitting reactor.

9. A process for splitting protein materials as specified in claims 1-5, wherein the splitting is used to destroy gluten in a wheat meal.

10. A process for decontamination of bone meal from pathogenic protein agents by splitting protein materials as specified in claim 8, wherein the protein material is an animal by-product and the splitting occurs at 100° C.-160° C. at a pressure 1-6 bar for 30 120 min and the separated insoluble bone material is treated in the protein splitting reactor at 100° C.-160° C. at a pressure 1-6 bar for 15-45 min.
